(19) United States
(12) Patent Application Publication
(10) Pub. No.: US 2025/0236769 A1
(43) Pub. Date: Jul. 24, 2025

(54) HOT MELT ADHESIVE FORMULATIONS FOR FIBROUS SUBSTRATES

(71) Applicant: SAVARE I.C. S.R.L., Milan (IT)

(72) Inventors: Stefania ASSONI, Milan (IT); Alberto BUGANA, Milan (IT); Italo Corzani, Milan (IT)

(21) Appl. No.: 19/175,168

(22) Filed: Apr. 10, 2025

Related U.S. Application Data
(63) Continuation of application No. PCT/IB2023/059329, filed on Sep. 21, 2023.

(30) Foreign Application Priority Data
Oct. 12, 2022 (IT) 102022000021018

(51) Int. Cl. C09J 11/08; A61F 13/15; A61F 13/511; A61F 13/514; A61F 13/515; B32B 5/02; B32B 5/26; B32B 7/12; C09J 5/06; C09J 11/06; C09J 123/16; C09J 123/20

(52) U.S. Cl. CPC C09J 11/08; A61F 13/515; B32B 5/022; B32B 5/024; B32B 5/263; B32B 5/266; B32B 7/12; C09J 5/06; C09J 11/06; C09J 123/16; C09J 123/20; A61F 2013/15284; A61F 2013/1591; A61F 2013/51139; A61F 2013/51178; A61F 2013/51441; A61F 2013/51452; B32B 2255/02; B32B 2255/26; B32B 2262/062; B32B 2307/748; C09J 2400/266

(57) ABSTRACT

The present invention discloses novel hot melt adhesive formulations which exhibit an exceptionally good adhesion on fibrous substrates, both woven and non-woven ones. Furthermore the present adhesives exhibit an adhesion on said substrates which continues to be surprisingly high even when those fibrous substrates are made of very hydrophilic fibres, as cotton and other similar vegetable fibres, and even when said hydrophilic fibers, during their use e.g. inside a hygienic absorbent article, contact water and absorb large amounts of water or of other aqueous fluids, e.g. urine or blood, with an ensuing relevant swelling of the same fibres, said relevant swelling being capable of strongly weakening and even of destroying and mechanically breaking the adhesive bond, already formed under dry conditions.

### CROSS REFERENCE TO RELATED APPLICATIONS

This is a continuation application of PCT international application PCT/IB2023/059329, filed on Sep. 21, 2023, which claims the priority of Italian Patent Application No. 102022000021018, filed Oct. 12, 2022, both of which are incorporated herein by reference in their entirety.

### TECHNICAL FIELD OF THE INVENTION

The present invention refers to hot melt adhesive formulations which are able to strongly adhere on fibrous substrates, both woven and non-woven ones. The present adhesives in particular are able to adhere in a surprisingly good way and are able to withstand the forces that tend to weaken or break the adhesive bond, even when the fibers of said substrates are strongly hydrophilic and therefore can, in the presence of aqueous liquids, absorb high amounts of water and swell in a substantial way.

The present hot melt adhesives are especially suitable for being used in the construction and manufacturing of hygienic absorbent articles that contain, as it almost always happens, at least one fibrous substrate, said fibrous substrate being a woven or a non-woven substrate, and being its fibers natural fibers or synthetic ones.

### TECHNICAL BACKGROUND

Inside hygienic absorbent articles, the above-mentioned fibrous substrates, both woven or non-woven ones, and made both of natural or synthetic fibers, are very often assembled in more complex structures through the use of adhesives. Said structures are e.g. named “laminates”, and are formed by two or more layers that are bonded by a glue one to the other.

However, in a few cases, as for example is taught by EP 0924328A1, it is also possible to make multi-layer structures, having at least one layer formed by a fibrous substrate, woven or non-woven, by performing a localised melting, in selected points, of the substrate itself, said localised melting being obtained e.g. by the application of ultrasounds. However, it is evident that said technique is applicable only to synthetic fibres, made of thermoplastic polymers, and that it is not applicable to natural fibres that are essentially made of cellulose. Moreover, the partial and localised melting of at least one of the fibrous substrates generates at least two further problems. First of all, in those points where the thermoplastic fibres are melted, areas with a continuous structure and without any pore are formed. Therefore, in this way in those areas the fibrous substrate loses, at least locally, the natural porosity that is typical of all fibrous substrates; and with that porosity it loses, at least partially, the advantages that derive from it, like the breathability and the permeability to liquids. As a second disadvantage, the local melting generates areas of molten polymer that are very hard and stiff, which therefore also stiffen the whole laminate in a substantial way. Said stiffness might be acceptable in some industrial uses, for example in non-woven laminates that are used as bases for floor-carpets or laminates used as separators inside electric batteries; but it is not at all acceptable e.g. in articles like hygienic absorbent articles, where the high softness of the whole structure is a fundamental characteristic, highly appreciated by users.

Therefore, the construction of multi-layer laminates, in which at least one layer is a fibrous substrate, through the use of adhesives is the preferred solution in many fields and especially in the hygienic/sanitary field. Moreover, it is also evident that in this field, as well as in other similar fields, the quantity of used adhesive must be kept as low as possible. This not only for obvious economic reasons, but also because only if the adhesive is coated in a discontinuous way, and therefore in a limited quantity, it is possible both to keep the porosity of the fibrous substrate as well as to avoid a possible stiffening of the laminate due to an excessive and continuous layer of adhesive.

A part of the Prior Art, e.g. U.S. Pat. Nos. 4,069,822 and 4,147,580, seems to especially focus on identifying the best process for applying sufficiently small quantities of a hot melt adhesive (for avoiding to stiffen the fibrous substrate and to totally close its natural porosity) nonetheless also ensuring an adequately strong adhesive bond. For example, according to these patents, this result can be achieved by trying to bond preferentially only the free fibrils, that stick out from the plane of the woven or non-woven fibrous surface. Apart from all possible doubts about the effectiveness of such process per se, in these patents nothing is said about the chemical and compositional characteristics as well as about the physical and performance properties of the used adhesives. This is clearly a weakness of said Prior Art, because it is obvious that the strength of the formed adhesive bond depends on the physical and chemical properties of the used adhesive, much more than on the process by which it is applied.

A similar technique is also disclosed in U.S. Pat. No. 4,849,049, that again exclusively focuses on the application process, disregarding once more the chemical and physical characteristics that are required to adhesives to be used on fibrous substrates.

Also U.S. Pat. No. 5,360,504 applies similar concepts for the bonding of a substrate which, even if it is not a fibrous substrate but in the specific case a polyolefinic sponge, shares with fibrous substrates the common characteristic of a very high porosity, and therefore the need for the hot melt adhesive to physically penetrate, at least partially, inside the substrate's pores, in order to lock it in a sufficiently strong way. In this case, U.S. Pat. No. 5,360,504, besides teaching a certain application process, identifies in the melt viscosity of the thermoplastic adhesive-viscosity that is generically required to be lower than the enormous value of 800,000,000 mPa·s—the only parameter of the adhesive that is useful, according to the inventors, for ensuring its effectiveness in the disclosed usage. This approach to the problem can be criticised under several aspects; but, in particular, one can observe that said enormous melt viscosity (even if it is not specified either the method for measuring it and even the temperature, which can range from 140 F (60° C.) to 330 F (165.5° C.)) is most likely measured—as it generally happens for hot melt adhesives—according to the method ASTM D 3236-88 (the so called Brookfield viscosity). Therefore, it is most likely measured under a shear-rate different from zero, a condition that actually, as it will be shown later, is not representative of the real conditions under which a molten adhesive penetrates inside a porous substrate.

Finally, other Prior Arts, for example U.S. Pat. No. 5,626,912, make use of reactive adhesives for consolidating structures formed by fabrics and other fibrous substrates; this because a reactive adhesive, which is applied as a blend of its monomers or as low viscous oligormers, and that is then polymerised in place after its application, combines the advantage of the initial very low viscosity, that allows an easy penetration inside the fibrous substrate's pores, with a strong resistance to detachment after the polymerisation. However, it is clear that the use of reactive adhesives is not possible in many cases, both because of the process complications that the post-application polymerisation introduces in industrial production lines (e.g. a significant decrease in the line's speed), as well as because in many fields (e.g. the hygienic/sanitary field, the medical field, the field of items for food contact etc.) it is preferred to avoid the use of reactive adhesives owing to the fact that, even after polymerisation, they may still contain residues of unreacted monomers that often are highly toxic and/or carcinogenic.

Therefore, the existing Prior Art does not seem to have identified so far satisfactory solutions to the problem of making bonded structures (laminates), that contain at least one fibrous substrate, both woven or non-woven, through the use of non-reactive hot melt adhesives. In particular, it seems that none of these prior techniques has wondered about what it happens in these bonded structures when their fibrous substrates are made of highly hydrophilic fibers, e.g. cotton or other vegetable fibers, that during use can absorb even very high amounts of water or of other aqueous liquids, like urine or blood. This problem arises from the fact that the absorption of water (as it is better explained below) has an extremely negative, and often even destructive, effect on the great majority of adhesive bonds, both because of the chemical and physical effects deriving from the presence of liquid water per se, and of the mechanical effects of the very large swelling of the fibres, as a consequence of the absorption of water, a mechanical effect that tends to break the pre-existing adhesive bonds.

EP 3 858 933 discloses hot-melt adhesives comprising, as their fundamental component, at least one polymeric composition of butene-1, which is obtained through a metallocene catalytic system, and which has a bimodal structure/distribution. Said composition comprises less than 5% by weight of a viscosity modifier that is not solid at room temperature (i.e. at 23° C.) and optionally less than 5% by weight of a wax or of a mixture of polyethylene waxes, having a linear structure and a low Index of Polydispersity.

WO 2021/127 351 discloses a hot-melt adhesive formulation comprising at il 40% by weight of polybutene-1, synthesized through a metallocene catalytic system. Said polybutene-1 may be a homopolymer or a copolymer or a mixture thereof. It is requested that the presence of wax is in an amount of at least 15% by weight.

### SUMMARY OF THE INVENTION

The problem that the present invention aims at solving is to teach to formulate hot-melt adhesives that are able to strongly adhere on fibrous substrates, both woven and non-woven. The present novel adhesives are able to adhere in a surprisingly strong way in the dry state and are also able to retain a strong adhesion even when those fibrous substrates, owing to the high hydrophilicity of their fibers (e.g. cotton and other natural fibers) during their use, absorb high quantities of water or of other aqueous liquids, like blood or urine, in this way undergoing a considerable swelling of said fibres, which swelling is capable of mechanically breaking the adhesive bond that was formed in dry conditions.

Therefore, the hot melt adhesive according to the present invention are especially suitable for being used in the manufacturing of hygienic absorbent articles which contain at least one fibrous substrate, both woven or non-woven, and which are made both of natural or of synthetic fibres. In particular, according to what said above, the present adhesives are able to form and retain a surprisingly strong adhesive bond even in the case when said highly hydrophilic natural fibres (e.g. cotton and similar vegetable fibers), during their use inside hygienic absorbent articles, absorb large amounts of water or of other aqueous fluids, like urine or blood. In fact, in said conditions, in which the great majority of adhesives are unable to keep a strong adhesion on wet fibers and even more on fibres soaked with water, on the contrary the present hot melt adhesives are surprisingly able to retain a strong adhesion and remain bonded in an excellent way. In such a manner they show to be capable of withstanding, as it will be better explained later, both the chemical and physical action of water that tends to annihilate and to destroy the intermolecular forces that create the adhesion between the adhesive and the substrate, as well as of withstanding the very powerful mechanical action exerted by the fibres when they soak and swell with water, such mechanical action being so powerful to easily break into pieces the adhesive itself, in this way physically destroying the adhesive bond.

These novel and unexpected properties of the present adhesives, able to strongly adhere on fibrous substrates and of vigorously withstanding the debonding even when those fibres are soaked with water during their use, are achieved by selecting and formulating said hot melt adhesives according to the criteria specified in claim 1; and in particular selecting and formulating hot melt adhesives which have:
a Zero Shear Viscosity not greater than 10,000 mPa, s and preferably not greater than 6,500 mPa·s;
an Enthalpy of Fusion, after aging for five days at room conditions (see later for the definition of “aged conditions”) that is not greater than 30 J/g;
a Temperature of the Inflection Point of the Tan Delta diagram as a function of temperature, said Inflection Point being located around the Setting Point, as measured at Time Zero and in decreasing temperature according to the method described below, which is not higher than 95° C.

Therefore the present invention concerns the use of a hot melt adhesive formulation which exhibits:
a Zero Shear Viscosity not greater than 10,000 mPa·s and preferably not grater than 6,500 mPa·s, at 160° C.;
an Enthalpy of Fusion not grater than 30 J/g, after aging for five days;
a Temperature of the Inflection Point of the Tan Delta diagram as a function of temperature, not higher than 95° C., said Inflection Point being located around the Setting Point, as measured at Time Zero and in decreasing temperature, according to the method described herein;
said hot melt formulation further exhibiting:
Dry Peel strength, after aging for five days, not lower than 2.0 N per 50 mm width;
wet Peel strength after water absorption and after aging for five days, which differs from the corresponding Dry Peel Strength by no more than 80%;
wherein such Peel Strengths are measured according to the methods described herein,
in the manufacturing of absorbent hygienic articles, which contain at least one fibrous substrate, said fibrous substrate being either a woven or a non-woven one, and its fibres being either natural or synthetic ones, either hydrophobic or hydrophilic, wherein the said composition includes at least one wax or a mixture of waxes synthesised from ethylene and maleic anhydride or from propylene and maleic anhydride or C12-C40 hydrocarbon waxes, in their versions modified with carboxylic acid or alcoholic groups, said wax or waxes having a “Drop Melting Point” in the range from 40° C. to 170° C., as measured according to the ASTM D 127-87 method.

Said hot-melt adhesive formulation is also being characterised in that it shows
a Zero Shear Viscosity at 160° C. not greater than 10,000 mPa·s and preferably not greater than 6,500 mPa·s, as measured according to the herein described method;
an Enthalpy of Fusion, after aging for five days, not greater than 30 J/g, as measured according to the herein described method;
a Temperature of the Inflection Point of the Tan Delta diagram as a function of temperature, not higher than 95° C., said Inflection Point being located around the Setting Point, as measured at Time Zero and in decreasing temperature, according to the method described herein;
said hot-melt adhesive formulation being further characterised in that it has:
a Dry Peel Strength, after aging for five days, not lower than 2.0 N per 50 mm width;
a Wet Peel Strength after water absorption and after aging for five days, which differs from the corresponding Dry Peel Strength by no more than 80%;
wherein such Peel Strengths are measured according to the methods described herein, said hot-melt formulation for the adhesion to fibrous substrates having also:
a Yield Stress not lower than 0.1 MPa after aging for five days, as measured at a temperature of 37° C., according to the method described herein;
having a Toughness not lower than 0.5 MJ/m³ after aging for five days, as measured at a temperature of 37° C., according to the method described herein.

Said hot-melt adhesive formulations moreover preferably show also a First Crossover Temperature Tx of the rheological modules not higher than 75° C., as measured at Time Zero and in decreasing temperature, according to the method described herein.

In the following paragraphs the inventors will explain in more detail the possible reasons why the above-mentioned chemical-physical properties can lead to the surprisingly good behavior of the present hot-melt adhesives.

Therefore, the problem that the present invention aims to solve can be considered fully solved in an optimum and unexpectedly effective way, by a use according to any one of the preceding claims from 1) to 13); by hot-melt adhesive formulations having the characteristics of claim 14); by a bonded structure having the characteristics of claims from 15) to 17); and by an article having the characteristics of claims from 18) to 24). The other sub-claims disclose preferred embodiments.

### Definitions

The expressions “comprising” or “that comprise(s)” are used herein as open-ended terms, that specify the presence of what in the text follows said terms, but that does not preclude the presence of other ingredients or features, e.g. elements, steps, components, either known in the art or disclosed herein.

The expression “polymer(s)” is used herein according to the definition given in the document issued by ECHA—European Chemical Agency—December 2017 edition—and titled “How to decide whether a substance is a polymer or not and how to proceed with the relevant registration”. Hence, in the present invention we define as a “polymer” any chemical substance that contains more than 50% by weight of “polymeric molecules”; where said “polymeric molecules” are in turn defined as those molecules that contain at least three base units (monomeric ones or more complex) that are bound to a fourth unit, that can be equal or different from the first units. Therefore, said polymeric molecules contain in total at least four base units, that can be monomeric units or more complex ones (when e.g. the base unit is, in its turn, composed by two or more monomers as it happens in condensation polymers). The expression “polymer(s)” comprises both polymeric molecules formed by just one type of base units/monomer (homopolymer) as well as by multiple different types (copolymer).

In a similar way, the expression “oligomer(s)” means herein a chemical substance that contains more than 50% by weight of “oligomeric molecules”; where said “oligomeric molecules” contain less than three base units (monomeric ones or more complex) bound to another unit that can be equal or different from the first units. Also, the expression “oligomer(s)” comprises both oligomeric molecules formed by just one type of base units/monomer (homo-oligomer) as well as by multiple different types (co-oligomer).

More specifically, the expression “homopolymer(s)” is used herein according to the definition given by IUPAC (International Union of Pure and Applied Chemistry) in the article “GLOSSARY OF BASIC TERMS IN POLYMER SCIENCE”, published in “Pure and Applied Chemistry”, Vol. 68, No. 12, pp. 2287-231 1, 1996. Therefore, the expression “homopolymer(s)” means herein a polymer that is synthesised from just one type of monomer. Still according to the same reference, the expression “copolymer(s)” means in the present invention (unless it is specifically indicated a different meaning) not only a polymer in the chemical composition of which two different monomers are used, but also polymers in the chemical composition of which three, four, five or more different monomers are used. According to the above-mentioned reference, when one wants to emphasise the number of different comonomers that form a certain copolymer, one can also use, as an alternative, the expressions “bipolymer”, “terpolymer”, “quaterpolymer” and so on.

The expression “First Crossover Temperature of the rheological Moduli”, often indicated with the symbol Tx, which is also sometimes named “Rheological Setting Point” or “Temperature of Rheological Setting”, refers, in a rheological diagram in which are measured, as a function of temperature, the Viscous Modulus G″, the Elastic Modulus G′ and their ratio Tan Delta, the highest temperature at which the two Moduli cross (and where therefore the value of Tan Delta is equal to 1) in the field of temperatures above room temperature (i.e. above 23° C.). Said rheological diagram, when measured at Time Zero and in conditions of decreasing temperature, mimics very well the phenomena that occur between the adhesive and the substrate during the real process of application from the molten state of a hot melt adhesive and in the creation of the adhesive bond, during the slow spontaneous cooling and solidification of the adhesive. In particular, the “First Crossover Temperature of the rheological Moduli” or, with an equivalent expression, the “Rheological Setting Point”, identifies the temperature at which the hot melt adhesive, applied from the molten state on a substrate, begins to create the final adhesive bond in the solid state.

The expression “Crossover Modulus” and the corresponding symbol Gc, refer, still in the above-mentioned rheological diagram, to the absolute value (by definition identical for both Modules) expressed in Pa or in MPa, that the elastic modulus G′ and the viscous Modulus G″ of a certain material, have at its Temperature of Rheological Setting Tx.

The Zero Shear Viscosity is defined in Rheology as the asymptotic constant value (Plateau) to which the viscosity of a liquid polymeric system (e.g. a molten polymer or a polymer solubilised in a solvent) is tending when the applied Shear Rate tends to zero, in an experimental curve that expresses the apparent viscosity as a function of the applied Shear Rate. For the present invention, the liquid polymeric systems the Zero shear Viscosities of which are measured are the molten thermoplastic adhesives at the temperature of 160° C. This parameter is clearly defined e.g. in the article “On finding the zero-shear-rate viscosity of polymer melts” by M. T. Shaw published in “Polymer Engineering and Science”—Vol. 61—February 2021. As it is well known, this rheological parameter is proportional to the Weight Average Molecular Weight Mw of the polymer under test; and it is measured and calculated according to the teachings of the article “Getting the zero shear viscosity of polymer melts with different rheological tests” by J. Lauger and M. Bernzen, published in “Annual Transactions of the Nordic Rheology Society”, Vol. 8. 2000, pages. 159-1 More precisely, among the various rheological methods described in said article for calculating and measuring said physical parameter, in the present invention the Zero Shear Viscosity is measured according to the method “Flow curve at low shear rates” described in said article, and it is expressed in mPa·s.

On the contrary the dynamic viscosity of an adhesive in the molten state at a certain temperature and under a Shear Rate different from zero, which in the Industry is also called the Brookfield Viscosity, is herein measured according to the method ASTM 3236-88 and it is again expressed in mPa·s.

The Enthalpy of Fusion, the Enthalpy of Crystallisation, and the Glass Transition Temperature of a certain material, are herein measured by Differential Scanning calorimetry (DSC). All these DSC measures, both in crystallisation cycles i.e. in decreasing temperature, and in melting, i.e, in increasing temperature, are performed according to the standard Method ASTM D3417-99. Said cycles are performed between +180° C. and −70° C. (or vice versa) at a rate of temperature change—both in increasing and in decreasing—equal to 10° C./minute. When, for a certain material, the DSC Fusion or Crystallisation diagrams show two or more peaks (in case even partially overlapping), it is herein defined as the “Peak Melting Temperature” of that material or as the “Peak Crystallisation Temperature” of that material, the Peak Temperature of the peak that has the greatest area, and hence has the greatest Enthalpy. The overall value of the Enthalpy of Fusion or of Crystallisation is calculated as the sum of all the peaks of fusion or of crystallisation detected in the DSC test. The Enthalpies of Fusion and of Crystallisation are expressed in J/g and are given by the area (obtained by integration) of the peak or of the sum of the areas of the peaks that appear in a certain DSC cycle.

“Room Temperature”, unless differently specified, means a temperature equal to 23° C.; and “Room Conditions” means the conditions of an environment at controlled temperature and relative humidity, i.e. at 23° C. and at 50% relative humidity.

Unless differently specified, all the rheological parameters reported in the present invention, for example the elastic Modulus G′ of a certain adhesive, are measured at a frequency of 1 Hz, in a rheological test in decreasing or increasing temperature, between +170° C. and −20° C. (or vice versa), in which the rate of change of the temperature is equal to 3° C./minute.

Because a few materials used in the present invention change some of the their properties, for example the quantity and morphology of their crystalline factions (and hence vary their mechanical properties and their Enthalpies of Fusion) as a function of the time elapsed the moment from of their solidification from the molten state, in the present invention said properties that can change with time are distinguished in “Properties at Time Zero” and in “Aged Properties” at a certain number of hours or days, typically five days, that have passed after their solidification from the molten state. Therefore, a certain property “at Time Zero” (e.g. an Enthalpy of Fusion at Time Zero), called also an “initial property” or a property “under initial conditions”, means that said property is measured at 23° C. (unless a different temperature of measurement is specified) and at 50% relative humidity, at a time that is not longer than 120 minutes from the moment when the material has solidified from its molten state. On the contrary, a certain property “aged at five days” or “in aged conditions” means that said property is measured at 23° C. (unless a different temperature of measurement is specified) and at 50% relative humidity, after five days from the solidification of the tested material from its molten state. During these five days of aging the tested material is kept in a climatic room at 23° C. and 50% relative humidity.

The tensile properties, called also “mechanical properties under tension” of a certain material, for example an adhesive formulation, like the curve Stress versus Elongation at Break, and their deriving parameters like the Peak Stress, the Yield Stress, the Stress at Break. The Elongation at Break and the Toughness are herein measured at 37° C. and 50% relative humidity, according to the method described below. In particular, in a curve Stress versus Elongation of a certain material, the Yield Stress is defined as the stress at the Yield Point, which is in turn identified as that point of said curve in which the material ceases to have an “elastic” behaviour (i.e. in which the applied stress and the recorded elongation are proportional) and starts to have a “plastic” deformation. Practically, said Yield Point coincides with the point where the curve Stress versus Elongation ceases to have a straight shape and bends showing a curvilinear shape.

The Toughness of a certain material is numerically expressed by the total area, calculated by integration, that is subtended by the curve Stress versus Elongation. It expresses the specific energy that is required for mechanically breaking a certain material and is expressed in MJ/m³.

The rheological and tensile properties, as well as the curve Stress versus Elongation, are herein measured with a rotational Rheometer Ares G2, supplied by TA Instruments. For measuring the tensile properties and for determining the curve Stress versus Elongation, said rheometer is equipped with an accessory tool called Extensional Viscosity Fixture (EVF). The rheometer is equipped also with a controlled temperature space (FCO) that allows to perform tests at a controlled temperature between −50° C. and +250° C.

In the test for the curve Stress versus Elongation, the tested sample is extruded, by a lab-coater for hot melt adhesives, at the temperature of 160° C., on silicone paper as a continuous strip having a width of 50 mm and a thickness of 0.2 mm. The extruded adhesive is aged for five days in a climatic room at 23° C. and 50% relative humidity before performing the test. After five days of aging, the samples for the test, with a length of 100 mm each are cut from this continuous strip of aged adhesive. Said samples are tested by using the EVF tool at the temperature of 37° C. and at the rotation frequency of the extension-roll equal to 1 turn/s. These parameters are selected in a way to mimic as much as possible the real conditions under which the adhesive needs to work inside a hygienic absorbent article, at the body temperature of 37° C. and at a high strain rate equal to 1 turn/s for simulating the very quick swelling of the cotton fibers that, when absorbing in a very fast way the water of the aqueous body fluids (urine, menstrual blood), rapidly swell and powerfully stretch the adhesive attached on said fibers.

The substantive “compatibility” and the adjective “compatible”, referred to the mutual blends of the ingredients of the present hot-melt adhesive formulations, and in particular to the blends of two or more polymers, are herein considered in the meaning defined in the “IUPAC. Compendium of Chemical Terminology”-2nd Edition-1997. I.e. a blend is “compatible” when it shows macroscopically uniform physical properties, independently from the fact that it is formed by “miscible” blends (i.e. that it shows just one Glass Transition Temperature, Tg) or by “immiscible” blends (i.e. two or more Tg's). In particular, the present invention considers as “compatible” all those blends that, when kept in the molten state at 170° C. for 72 hours, do not show any visual de-mixing in two or more layers/phases.

“Polydispersity Index” or “Molecular Weights Distribution Index” or “PDI” refers to a measure of the distribution of the molecular weight in a certain polymer. It is defined as the ratio between the Weight Average Molecular Weight Mw, and the Number Average Molecular Weight Mn: PDI=Mw/Mn. Greater values of PDI correspond to broader distribution curves of molecular weights and vice versa. Even for compatible blends of polymers it is possible to define an average Mw, an average Mn and therefore a global “Index of Polydispersity” as defined in the case of single polymers. Mw, Mn and their ratio Mw/Mn=PDI, are measurable e.g. by Gel Permeation Chromatography (GPC).

“Open Time” of an adhesive refers, especially for a hot-melt adhesive, to the interval of time during which, after its application from the melt on a first substrate, the adhesive is able to form sufficiently strong adhesive bonds for the intended use, with a second substrate that is brought into contact under moderate pressure with the first one. It is evident that too short open times may make difficult-to-manage the application of an adhesive and the formation of sufficiently strong bonds. The open time of a holt-melt adhesive may be measured according to the test method ASTM D 4497-94, with the following conditions for the hot-melt adhesives disclosed herein:
Coating temperature of the adhesive film: 170° C.
Thickness of the adhesive film: 1 mm

“Ring & Ball Softening Point” or “Ring & Ball Softening Temperature” refers to the softening temperature of a material, measured according to the Method ASTM D 36-95.

Just for waxes, the Softening Point (known in this case also as “Dropping Point” or as “Drop Melt Point”) is measured according to the Method ASTM D 3954-94.

The “Needle Penetration” of an adhesive is a measure of its softness. It is generally expressed as tenths of millimeter, dmm, and it is herein measured according to the Method ASTM D1321-04.

The overall Adhesive Strength or “Peel Strength” is defined as the average strength per unit of width needed to separate two substrates, bonded by the adhesive under test. It is measured through a separation test made at a controlled and constant speed, and under a controlled and constant debonding angle. It is herein measured according to the Method ASTM D 1876-01, separating the two substrates under a debonding angle of 180 degrees, by applying a separation speed of the two substrates equal to 150 mm/minute, that means that the testing dynamometer is actually moving at a speed of 300 mm/minute. The two substrates used herein are a polyethylene film, with a basis weight of 15 g/m², supplied by Poligof (Italy), that is glued to a spunlace non-woven 100% HyDry Cotton, made with cotton fibers and with a basis weight of 35 g/m², supplied by Glatfelter (USA). The adhesive under test is melted at 160° C. and it is then applied by spraying or by slot-coating on the cotton substrate at the basis weight of 8 g/m². and immediately put in contact and adhered with the polyethylene film. The bonded laminates are then aged for five days in a climatic room kept at 23° C. and 50% relative humidity. At the end of such aging, the samples are tested for their Peel Strength. This measurement is made by recording the strength needed for separating the two glued substrates on a width of 50 mm, according to the recommendations of the above-mentioned ASTM method, and working at 23° C. and 50% relative humidity. The Peel Strength measured according to the above-described method is herein defined as the “Dry Peel Strength” or as the Peel Strength “in dry conditions”.

However, because the Peel Strength can significantly change in the presence of liquid water, as it will be better explained in the following paragraphs, in the present invention we distinguish between the Peel Strength under dry conditions or Dry Peel Strength and the Peel Strength “after absorption of water”, called also “Wet Peel Strength” or the Peel Strength “under wet conditions”. In fact, the presence of liquid water can cause dramatic variations of the adhesive strength, especially when a fibrous substrate is made of highly hydrophilic fibres, e.g. cotton fibres and other similar vegetable fibres, that can absorb in use very large amounts of water and that, as a consequence, swell very much.

For measuring such Peel Strength “after absorption of water” or “Wet Peel Strength”, the following method is used: still under room conditions, i.e. at 23° C. and 50% relative humidity, one takes samples of laminate that are fully identical to the ones used also for measuring the corresponding Dry Peel Strength. On each sample one pours with a calibrated syringe, on the cotton non-woven, 5 ml of distilled water, that is then manually distributed in a uniform way on all the sample's area. One waits for 300 s to ensure that the cotton has completely absorbed all the water and that its fibres are fully swollen. At this point, the Peel Strength “after absorption of water” or “Wet Peel Strength” is measured, still by following the recommendations of the ASTM Method D 1876-01 and in the same conditions described above.

“Hygienic absorbent articles” refer to devices and/or methods concerning disposable absorbing and non-absorbing articles, that comprise diapers and undergarments for incontinent adults, baby diapers and bibs, training pants, infant and toddler care wipes, feminine catamenial pads, interlabial pads, panty liners, pessaries, sanitary napkins, tampons and tampon applicators, wound dressing products, absorbent care mats, detergent wipes, and the like.

Other less usual parameters, that are measured according to specific methods, will be defined later, together with the detailed description of the methods for measuring them.

### BRIEF DESCRIPTION OF FIGURES

In the attached Figures:

FIG. 1 presents a diagram that shows the identification of the Inflection Point of the experimental curve of Tan Delta vs. Temperature for Comparative Example 1 and

FIG. 2 presents a diagram that shows the identification of the Inflection Point through the value equal to zero of the second derivative of the function of Tan Delta vs. Temperature for Comparative Example 1

### DETAILED DESCRIPTION OF THE PREFERRED EMBODIMENTS AND OF THE MAIN COMPONENTS AND PROPERTIES OF THE HOT MELT ADHESIVES ACCORDING TO THE PRESENT INVENTION

The hot melt adhesive formulations according to the present invention show an exceptionally good adhesion on fibrous substrates, both woven and non-woven ones. In particular, these adhesives show an adhesive strength that remains surprisingly high even when said fibrous substrates are formed by strongly hydrophilic fibres, such as cotton and similar vegetable fibres, and even when said hydrophilic fibres, during their use, come into contact and absorb large quantities of water or of other aqueous fluids, such as urine or blood.

Even just a strong adhesion on a perfectly dry fibrous substrate is a quite difficult goal to achieve, because of several reasons, as already partially seen in examining the Prior Art. These difficulties in forming a strong adhesive bond even on dry fibrous substrates are due, for example, to the unevenness of the fibrous surface that does not allow a uniform distribution of the adhesive; to the reduced area available for the contact with the adhesive, as a consequence of the presence of holes among the fibres; to the presence of free fibrils which are not strongly fastened to the rest of the fibres and that can be easily pulled out etc.

Said adhesion becomes even more difficult when these fibrous substrates are wet or, even worse, when they can absorb significant quantities of water during their use, due to the strong hydrophilicity of their fibres. In fact, it is well known in the technology of adhesives that a good adhesion on wet substrates is one of the most difficult results to be achieved, unless one uses extremely peculiar adhesives, like for example reactive hydrogels, based on water and acrylic monomers and crosslinked with UV radiations. Such peculiar adhesives, however, do not fall into the possible uses at which is aimed the present invention for several reasons: e.g. very high cost, need of special packaging to avoid dehydration, technical difficulties in their production, and especially possible toxicity/carcinogenicity of the unreacted monomers etc.

Considering hot melt adhesives, it is well known that for the great majority of these adhesives the presence of liquid water, of wet substrates and of substrates that, due to their hydrophilicity, can absorb large quantities of water, in this way also greatly swelling, are among the worst possible enemies for achieving and maintaining a strong adhesion.

Water acts in a very negative way on adhesion, because of numerous and different mechanisms and phenomena: it opposes both the formation of strong adhesive bonds on already wet substrates, as well as it destroys existing strong adhesive bonds, already formed in the dry state, when such already glued dry substrates contact water and even worse when they can absorb large quantities of water, owing to their own hydrophilicity.

First of all, the presence of an extremely polar liquid substance like water tremendously changes the fundamental forces that, at an intermolecular level, are the basic cause of all adhesive bonds. In fact, the presence on the surface of a substrate of a layer of water, even at an extremely thin level, like a film of water with the thickness of just a few molecules, completely changes, owing to the powerfully polar nature of liquid water, all the chemical-physical and physical phenomena that concur to create the adhesive bond between the hot melt and the substrate. About the physical phenomena, this happens because a film of liquid water, even with the extremely thin thickness of a few molecules, creates what in the Science of Adhesion is known as a “weak layer”, i.e. a layer that immediately mechanically fails and breaks under even an extremely small applied stress, in this way causing the failure and breaking of the whole adhesive bond and of the whole macroscopic structure. Moreover, also from a chemical-physical standpoint, this happens because the very strongly polar nature of water changes and destroys all the elementary and fundamental attraction forces that exist between the adhesive and the substrate, like the van der Waals forces, the Dipole forces and the forces generated by Hydrogen bonds. This extremely negative action on adhesion, caused by the presence of liquid water on the surface of a substrate, is the first cause making it so difficult, and in many cases even practically impossible, to adhere on a wet substrate by using standard thermoplastic adhesives. This is also the reason why a strong adhesive bond, that has been previously formed between an adhesive and a substrate in the dry state, can be greatly weakened or even fully destroyed when the bonded structure contacts liquid water. For fibrous substrates, this is true for substrates which are made of both hydrophobic or hydrophilic fibres.

However, in case the substrate is made of hydrophilic fibres, and especially of strongly hydrophilic fibres, like all natural fibres as cotton and similar ones, an additional negative phenomenon occurs, a phenomenon that can have even more dramatically destructive effects even on a very strong adhesive bond between an adhesive and a substrate, said bond having been previously formed in the dry state.

When natural fibres, like cotton and similar vegetable fibres, or also artificial fibres derived from vegetable raw materials like rayon, contact liquid water, they are able to absorb surprisingly huge quantities of water, especially when (as it often happens for textile fibres) these fibres have undergone processes aimed at improving some important properties, like e.g. the so called process of mercerisation.

For example, under these conditions, cotton can absorb up to almost twelve times its own dry weight of liquid water (about 1,200% of its dry weight) as e.g. shown in the article “Analysis of Water Absorption of Different Natural Fibers”, published in Journal of Textile Science and Technology—Vol. 7 No. 4, November 2021; and also most other cellulosic fibres behave in a similar way.

Besides the already seen negative effect that a wet surface exerts on an adhesive bond, an additional effect, that is even much more dramatically negative for the survival of said bond, is therefore created by the very strong swelling that the absorption of such enormous amounts of water causes in hydrophilic fibres like cotton.

Said swelling by volume can be calculated to be equal to several times the initial dry volume of the same fibres; and because the swelling force is a “hydraulic force”, it is extremely powerful. Therefore, besides the negative effects on the adhesive strength, this huge swelling of hydrophilic fibres, like cotton and similar ones, has a further really destructive mechanical effect of the adhesive itself. In this way, the adhesive can be actually d crumbled by the extremely powerful hydraulic force generated by the strong swelling of the fibres after their contact with liquid water.

The hot melt adhesives according to the present invention are first of all capable of giving very strong adhesion on dry fibrous substrates, both woven and non-woven, and made both of hydrophobic (like synthetic polymeric fibres) or of hydrophilic fibres (like natural fibres, such as cotton and similar vegetable fibres, or as artificial fibres derived from vegetable raw materials, as Rayon, Lyocell and similar).

Moreover, in a fully surprising and unexpected way, the present hot melt adhesives are also capable of creating and maintaining a very strong adhesive bond on the mentioned fibrous substrates, even when said substrates are wetted, and even when said substrates (when they are strongly hydrophilic) contact, during their use, copious quantities of liquid water or of other aqueous body fluids, as urine and blood, absorbing strong amounts of said water and, as a consequence, swelling in a relevant way.

Hence, the present hot melt adhesives are especially suitable for being used in the manufacturing of absorbent hygienic articles, which in most cases contain at least one fibrous substrate, said fibrous substrate being both a woven or a non-woven one, and its fibres being both natural or synthetic ones, both hydrophobic or hydrophilic. In a particular way, the hot melt adhesives disclosed herein give surprisingly good results when said fibrous substrates, both woven or non-woven, are made of very hydrophilic natural or artificial fibres, for example cotton and other similar cellulosic fibres, which during their use can absorb very large quantities of aqueous fluids, like urine or menstrual blood.

Without being for this linked to any theory, we reasonably believe that all these surprisingly positive phenomena can be interpreted in the following way.

First of all, about the optimum adhesion shown on dry fibrous substrates, one can reasonably assume the following. As it is well known by all persons having an average expertise in the Science and Technology of adhesives, the overall adhesive strength that is formed between an adhesive and a substrate, is given by the sum of at least two fundamental contributions: the already mentioned attractive intermolecular forces (van der Waals forces, dipole forces, forces generated by Hydrogen bonds) and, in addition, the mechanical interlocking between said substrate and the adhesive, of course when the substrate has on its surface some unevenness and roughness, or even better has some pores and holes. In the case of fibrous substrates, because of their very high porosity and the many empty spaces among their fibres, it is obvious that a good mechanical interlocking between the adhesive and the fibrous substrate, is formed when the adhesive can penetrate, at least partially, into the holes/spaces among the fibres, even surrounding more or less completely the single fibres. This phenomenon has therefore a very special importance for creating a strong adhesive bond even on a fully dry fibrous substrate. The adhesives disclosed by the present invention are capable of penetrating, at least partially, inside the spaces that exist among the fibres, in this way forming with them particularly effective mechanical interlocking, and therefore an excellent adhesive strength still in the dry state, thanks also to two properties which are herein selected in a peculiar way.

First of all, the adhesives disclosed herein have a Zero Shear Viscosity that is not greater than 10,000 mPa·s and preferably is not greater than 6,500 mPa·s at 160° C., wherein said temperature of 160° C. has been chosen because it is a typical average temperature used in the process and application of hot melt adhesives. It is intuitive that a relatively low viscosity, in the molten state and at the temperature of application, makes easier and favours the physical penetration of the molten adhesive inside the pores of a porous substrate or inside a substrate that has rather large empty spaces, like a typical fibrous substrate, both woven or non-woven. However, the inventors of the present invention have found that in this case it is not correct to consider, as it often happens for hot melt adhesives, the dynamic melt viscosity known as “Brookfield viscosity” that is measured according to the method ASTM D-3236-88, under a Shear Rate different from zero. This is conceptually wrong, because the molten adhesive, as soon as it is extruded out of the extruding die and is coated on the fibrous substrate, is suddenly subjected to a Shear Rate equal to zero. Consequently, the correct physical property of the molten adhesive that first of all controls its ability to more or less penetrate into the holes of a fibrous substrate is actually its Zero Shear Viscosity. As it is known, in Rheology this property is expressed in mPa·s and it is defined as the constant asymptotic value (plateau) to which is tending the viscosity of a molten polymeric system, when the applied Shear Rate tends to zero, in an experimental curve that reports the apparent viscosity of the molten polymeric system (in our case of the molten adhesive at 160° C.) as a function of the applied Shear Rate.

The inventors found that excellent adhesive strengths on fibrous substrates are achieved when the Zero Shear Viscosity at 160° C. of the present hot melt adhesives is not greater than 10,000 mPa·s and preferably is not greater than 6,500 mPa·s. For the present invention, this property is measured and calculated according to the method “Flow curve at low shear rates” described in the article “Getting the zero shear viscosity of polymer melts with different rheological tests” by J. Lauger and M. Bernzen, published in “Annual Transactions of the Nordic Rheology Society”, Vol. 8. 2000, pages 159-162.

Moreover, the inventors have found that, in addition to the relatively low value needed for the Zero Shear Viscosity at 160° C., also at least two other rheological parameters of the molten adhesive play a fundamental role in ensuring an excellent adhesion, already in the dry state, favouring in this way the penetration of the adhesive inside the holes and in the empty spaces among the fibres, in this manner creating a strong mechanical interlocking between the adhesive and the fibrous substrate. These two further rheological parameters are:
the already defined “First Crossover Temperature” of the rheological moduli Tx, that is also called the “Temperature of Rheological Setting” or also the “Rheological Setting Point”;
and an additional rheological parameter that is called the “Temperature of the Inflection Point of the Tan Delta diagram as a function of temperature, located around the Setting Point”.

The parameter Tx or “First Crossover Temperature” of the rheological moduli has been already illustrated and defined above. This temperature, when it is measured, as done herein, at Time Zero and at 1 Hz, in decreasing temperature between +170° C. and −20° C., at a sufficiently small cooling rate as 3° C./minute, mimics very well the phenomena that happen between the adhesive and the substrate in the real process of application from the molten state of a hot melt adhesive and the ensuing creation of the adhesive bond, during the slow spontaneous cooling and solidification of the adhesive. In particular, said value of Tx at Time Zero identifies the point where the molten adhesive, that is spontaneously cooling after having been extruded and coated on the substrate, starts to “rheologically” solidify. I.e., over said value of temperature Tx, the Viscous Modulus G″ of the molten adhesive is numerically greater than its Elastic Modulus G′, and therefore the adhesive is capable of spontaneously flowing and hence penetrate in a sufficient way inside the pores and the empty spaces of the fibrous substrate. On the contrary, below said temperature Tx, the G′ of the adhesive is greater than its G″ and so the adhesive is no more able to flow and penetrate. For the hot melt adhesives according to the present invention, the inventors found that, in order to have an excellent adhesion on fibrous substrates, it is preferable that the present adhesives have a Tx which is not greater than 75° C.

However, the inventors have also surprisingly observed that an even more important influence than the one of Tx, for ensuring a strong adhesion of the present adhesives on fibrous substrates, is given by the so called “Temperature of the Inflection Point of the Tan Delta diagram as a function of temperature”, located around the Setting Point. Like Tx, also this parameter is measured in a rheological experiment and diagram, performed at Time Zero, in decreasing temperature at the cooling rate of 3° C./minute, between the temperatures of +170° C. and −20° C., and at the straining frequency of 1 Hz. More precisely, said Temperature of the Inflection Point of the Tan Delta diagram is identified in the following way. In the above described rheological diagram, one draws the curve of Tan Delta as a function of temperature, in a range of temperatures around Tx, i,e, around the temperature at which Tan Delta is equal to 1 and at which the adhesive is solidifying by cooling from its molten state. Said considered range of temperatures around Tx is comprised between the temperature at which Tan Delta is equal to 0.5 and the temperature at which Tan Delta is equal to 10. In this range of temperatures and of values of Tan Delta, the experimental curve that represents Tan Delta as a function of temperature, shows an inflection point, i.e. a point where the curvature of said curve changes its sign and direction. Said inflection point in the curve of Tan Delta and the corresponding temperature can be easily identified visually. As an alternative to the visual identification of said inflection point, one can also proceed in the following way: the experimental points in the curve of Tan Delta as a function of temperature are interpolated with a mathematical function, expressed by an equation of the third order, so that its correspondent Coefficient of Determination “R squared” is not lower than 0.95. From the equation, one can calculate the point where the second derivative of the function is equal to zero and changes its sign. The temperature corresponding to that point is the temperature of inflection in the curve. If for all the above-described rheological tests one uses a rotational rheometer ARES G2, supplied by TA Instruments, as herein recommended, said apparatus is equipped with a TRIOS software. Said software is able to automatically calculate the function which interpolates in the best way the experimental points of Tan Delta; and this software can also calculate the first and second derivatives of said function. In this case, the temperature corresponding to the inflection point of Tan Delta is immediately identified by looking at which temperature the second derivative of the curve Tan Delta versus Temperature, is equal to zero.

The temperature of said inflection point is herein called the “Temperature of the Inflection Point of the Tan Delta diagram as a function of temperature located around the Setting Point”, because the corresponding point lies in a range around Tx (Tan Delta=1), and more precisely between the temperature corresponding to Tan Delta=0.5 and the one corresponding to Tan Delta=10, in a range of temperatures above room temperature. Said Inflection Point, the temperature of which does not necessarily coincide with Tx, and that is often higher than Tx, has been surprisingly found to be an even more sensitive and important parameter, more than Tx, for determining a good or a bad adhesion on fibrous substrates of the hot melt adhesives disclosed in the present invention. Without being for this linked to any theory, one can suppose the following: around that Inflection Point one can observe a very quick variation of the numerical value of Tan Delta, a variation that can even be equal to an order of magnitude and even more (i.e. ten times or more) for a variation in temperature as low as about 10° C. At the same time, also the Elastic Modulus G′ shows a corresponding and similarly fast increase. These strong variations in the values of Tan Delta and of G′ in a limited range of temperatures around the Inflection Point of Tan Delta, are generally significantly larger than the variations of Tan Delta and of G′ that can be observed around the Rheological Setting Temperature Tx. This can be interpreted by considering that actually, even above Tx, the molten adhesives begin already to partially solidify, becoming in this way so viscous and semi-solid that it becomes for them difficult to penetrate, even partially, inside the holes of a fibrous substrate; and therefore, even slightly above Tx, they become already unable to give a good adhesion on said substrate. Accordingly, the inventors found that, in order to obtain an excellent adhesion on a fibrous substrate, the hot melt adhesives according to the present invention must have a Temperature of the Inflection Point of the Tan Delta diagram as a function of temperature, located around the Setting Point, that is not greater than 95° C., when measured at Time Zero in a rheological test in decreasing temperature between +170° C. and −20° C., at 1 Hz and at a cooling rate of 3° C./minute.

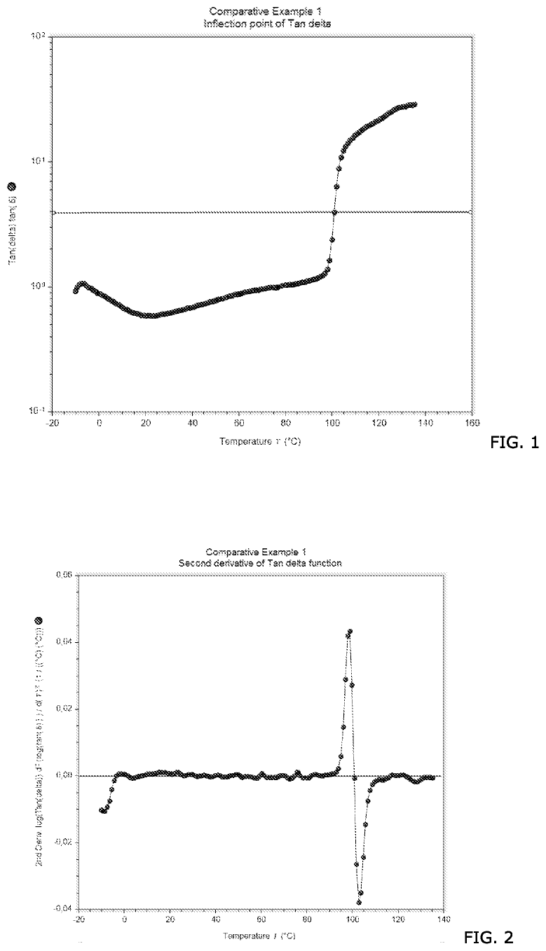

For clarity's sake, the enclosed FIGS. 1) and 2) practically show the identification of the inflection point in the experimental curve of Tan Delta (FIG. 1) and the identification of the inflection point through the value equal to zero of the second derivative of the function Tan Delta (FIG. 2) for the below described Comparative Example 1).

The above discussed chemical-physical and rheological parameters ensure an excellent adhesive strength on fibrous substrates, for example inside multi-layer laminates, already when said laminates are perfectly dry.

However, these parameters are not sufficient per se for allowing the adhesive having said characteristics, to keep a good adhesion even when said substrates and laminates contact liquid water or aqueous fluids, e.g. during the use inside hygienic absorbent articles, which contain fibrous substrates or laminates bonded with said adhesives. This is even truer in the case in which the fibres of said substrates are natural fibres, like cotton and similar ones, which are strongly hydrophilic and can absorb surprisingly huge quantities of water and swell consequently their volume by many times. In fact, as said, the presence of liquid water, besides weakening the elementary intermolecular forces on which adhesion is based, causes also, if fibres swell, a real mechanical action of detachment and of physical breaking and crumbling if the adhesive itself, which is coated on said swollen fibres.

In these cases, the inventors found that a surprisingly good adhesion can be retained even on highly hydrophilic fibres, like cotton and similar ones, and even in the presence of large quantities of liquid water and of a strong swelling of these fibres through the absorption of water or of aqueous fluids, provided that the hot melt adhesives, disclosed in the present invention, show, besides the already chemical-physical and rheological parameters described above, also the two additional parameters illustrated here below. More precisely:
a Yield Stress, measured after aging of five days, which is not lower than 0.1 MPa;
a Toughness, as measured again after five days of aging, that is not lower than 0.5 MJ/m³. Said Toughness is numerically expressed by the total area subtended by the curve Stress versus Elongation, in an experiment of Stress versus Elongation to Break of the tested adhesive, and as already said, it expresses the specific energy needed for physically crumbling the adhesive.

As mentioned, the tensile properties and the curve Stress versus Elongation to Break of the hot melt adhesives according to the present invention, are measured at 37° C. according to the previously described method.

Without being for this linked to any theory, we believe that what has been discovered in the present invention can be reasonably interpreted in the following way. Besides the already seen parameters, that allow to form excellent adhesive and mechanical bonds with fibrous substrates already in the dry state, a sufficiently high Yield Stress and Toughness ensure that the adhesive bond is able to mechanically resist and survive in an excellent way, even when the substrate is wetted, and its fibres swell due to the absorption of large quantities of water.

In addition to all the mentioned parameters, also a sufficiently low crystallinity of the adhesive, as expressed by an Enthalpy of Fusion not greater than 30 J/g after aging for five days, is advantageous for both the phenomena, i.e. both for the formation of an excellent initial adhesive bond in dry conditions, and for allowing said adhesive to withstand and survive when the fibrous substrate contacts liquid water and strongly swells. In fact, it is well known that, for a good initial adhesion, substances with low crystallinity “tacky” than crystalline substances; while, in order to favour the survival of the adhesive bond even in the presence of liquid water and of swelling fibers, one can reasonably suppose that substances with a low crystallinity are less fragile, and that, when they are subjected to a mechanical stress in tension, they tend more easily to plastically deform rather than breaking. It is therefore possible to suppose that an adhesive which has the features described in the present invention is able to withstand the action of destruction of the adhesive bond by the water which swells the fibres, not only because said adhesive has a Toughness and a Yield Stress that are sufficiently high, but also because, owing to its low crystallinity, the adhesive is not fragile and does not crumble into pieces under the mechanical action of the swelling fibres that, by absorbing liquid water, increase their volume by many times.

### Typical Components of the Adhesive Formulations According to the Present Invention

The adhesive according formulations to the present invention, which exhibit the above-mentioned chemical-physical parameters, typically comprise a series of chemical components which are described below in greater details.

### Polymers

The formulations according to the present invention comprise at least one thermoplastic polymer as their main ingredient. More specifically, they comprise at least one polymer, which can be a homopolymer or a copolymer, or they comprise a blend of two or more polymers, which again can individually be homopolymers or copolymers and that are mutually compatible, according to the definitions of these terms that have been previously given. Said polymer or said blend of polymers can have various chemical natures. Homopolymers and copolymers which are particularly suitable for the present invention are, for example, the homopolymers of an olefin from C2 to C12 or of a diolefin from C4 to C12, as well as the copolymers among the same olefins and diolefins; the copolymers between an olefin from C2 and C12 and vinylic and acrylic monomers, like e.g. poly-ethylene-vinyl acetate, poly-ethylene-methyl acrylate, poly-ethylene-acrylic acid and so on; the styrenic block copolymers, both in their non hydrogenated and in their fully hydrogenated form; and blends thereof.

Among the mentioned polymers, particularly preferred are, for example, the homopolymers and copolymers of the olefins from C2 to C8, and the fully hydrogenated styrenic block copolymers, as styrene-ethylene-butylene-styrene or styrene-ethylene-propylene-styrene; and blends thereof. Example of trade marks of industrial polymers that are suitable for the present invention, are e.g. the polyolefinic copolymers sold by the Company Rextac under the same trade mark; the copolymers C2/C3/C4 sold by Evonik under the trade mark Vestoplast; the homopolymers and copolymers C4 and C2/C4 sold by LyondellBasell under the trade mark Koattro; the copolymers C2/C3 sold by ExxonMobil under the trade mark Vistamaxx; the polyolefinic copolymers sold by Synthomer under the trade mark Eastoflex; the polyolefinic copolymers sold by Clariant under the trade mark Licocene; the C3 homopolymers sold by Idemitsu under the trade mark L-MODU.

The hot melt adhesives according to the present invention comprise from 5% by weight to 99.5% by weight of a polymer, which can be a homopolymer or a copolymer, or of a blend of two or more polymers, which are mutually compatible, and which can individually be both homopolymers or copolymers.

### Tackifiers

In an embodiment of the present invention, the hot melt adhesive formulations disclosed herein comprise at least one tackifier, which has a Ring & Ball softening point between 5° C. and 160° C.

In general, the tackifiers which are comprised in the formulations of the present invention can be selected from aliphatic hydrocarbon tackifiers, and their partially or totally hydrogenated derivatives; aromatic hydrocarbon tackifiers and their partially or totally hydrogenated derivatives; aliphatic/aromatic hydrocarbon tackifiers, and their partially or totally hydrogenated derivatives; modified polyterpene and terpene tackifiers, and their partially or totally hydrogenated derivatives; Rosins, their esters, and their partially or totally hydrogenated derivatives; and mixtures thereof. The partially and fully hydrogenated hydrocarbon tackifiers, both aliphatic or aromatic or aliphatic-aromatic, are particularly preferred. It has been furthermore discovered that it is more suitable that the tackifying resins, comprised in the adhesive formulations according to the present invention, have a Ring & Ball softening point ranging from 70° C. and 135° C., preferably from 80° C. and 130° C. and even more preferably from 85° C. and 125° C.

Particularly preferred are the tackfying resins which are highly purified and which contain an extremely low amount of volatile impurities and residual monomers, like xylene, toluene, hexene, vinyl-toluene, indene etc. which contribute to generate malodours in the finished product and to decrease the thermal stability of these resins. Said volatile compounds are detected by ionic gas-chromatography with a headspace, by heating a sample of 3 g of the tested tackifier for 30 minutes at 190° C., with a headspace equal to 20 ml. In the present invention those tackifying resins which contain a quantity of volatiles impurities not greater than 5 ppm (parts per million) are particularly preferred, preferably not greater than 2 ppm and even more preferably not greater than 1 ppm. Industrial examples of such highly purified tackifiers, with a very low content of volatile impurities, are the tackifying resins sold by the Company Synthomer (USA) under the trade mark UltraPure.

In the embodiment of the present invention in which the hot melt adhesive formulations disclosed herein comprise at least one tackifying resin, they comprise from zero to 80% by weight of at least one tackifying resin or of a blend or two or more tackifying resins, preferably from 3% by weight to 75% by weight, and even more preferably from 5% by weight and 70% by weight, with reference to the total weight of the adhesive formulation.

### Plasticisers

In another embodiment of the present invention, the hot melt adhesive formulations disclosed herein also comprise one plasticiser, which is liquid at room temperature, i.e. typically at the temperature of 23° C., or a blend of two or more plasticisers, said blend being liquid at room temperature. Said plasticisers further lower the melt viscosity of the adhesive formulations and increase their tackiness.

The plasticisers that are suitable for being used in the present invention are, for example, selected from paraffinic mineral oils and naphthenic mineral oils and mixtures thereof; paraffinic and naphthenic hydrocarbons which are liquid at room temperature, and mixtures thereof; polyolefin oligomers which are liquid at room temperature, and copolymers thereof, such as oligomers derived from ethylene, propylene, butene, iso-butylene, copolymers thereof, and mixtures thereof; plasticisers which are liquid at room temperature formed by esters, such as phthalates, benzoates, sebacates; natural and synthetic fats; vegetable oils; and mixtures thereof. Among the mentioned suitable plasticisers, the mineral oils, both paraffinic or naphthenic, and their blends; the poly-iso-butylenes, the synthetic oligomers of poly-alpha-olefins, that are liquid at room temperature, which are also known under the acronym “PAO” i.e. poly-alpha-olefin, are particularly preferred. Said synthetic oligomeric plasticisers, liquid at room temperature and usable in the present invention, are synthesised from olefins from C2 to C20 and have typically a Numeral Average Molecular Weight Mn ranging from 150 to 15,000 g/mole, preferably from 200 to 10,000 g/mole and even more preferably from 400 to 6,000 g/mole. Said PAO plasticisers are fully saturated and can have a substantially paraffinic structure, both linear or branched. Liquid poly-alpha-olefins useful as plasticisers in the adhesive formulations of the present invention, are manufactured and sold e.g. by ExxonMobil under the trade marks SpectraSyn and Elevast; by Ineos unde the trade mark Durasyn; by Chevron Phillips under the trade mark Synfluid etc.

In the embodiment of the present invention in which the hot melt adhesive formulations disclosed herein comprise at least one liquid plasticiser, they comprise from zero to 40% by weight of at least one plasticiser which is liquid at room temperature, or of a blend or two or more plasticisers, said blend being liquid at room temperature; preferably from zero to 30% by weight and more preferably from zero to 15% by weight, with reference to the total weight of the adhesive formulation.

### Waxes

In a further embodiment of the present invention, the hot melt adhesive formulations disclosed herein comprise at least one wax or a blend of two or more waxes. Said waxes can be natural or synthetic waxes and have a Drop Melting Point in the range from 40° C. to 170° C., as measured according to the ASTM D 127-87 method. However, because waxes can easily contribute, even when they are present in small quantities, to significantly increase both the Temperature of the Inflection Point of the Tan Delta diagram as well as the Tx of the adhesive formulation, it is preferable that the waxes which are used d in the present adhesives have a temperature of the Drop Melting Point that is not too high. More precisely, it is advisable that, if the present formulations comprise only one wax, said wax has a Drop Melting Point, as measured according to the method ASTM D 127-87, that is not greater than 125° C. If, on the contrary, the present formulations comprise a blend of two or more waxes, it is preferable that said blend comprises at least 25% by weight of at least one wax Drop Melting Point of which is not higher than 125° C., as measured again according to the method ASTM D 127-87, said percentage of at least 25% by weight being referred to the overall weight of all the waxes comprised in the formulation.

Waxes that can be used in the present invention are for example hydrocarbon synthetic waxes, like paraffin waxes, and in particular waxes synthesised from C2-C10 olefins and mixtures thereof; C12-C40 hydrocarbon waxes, even in their versions modified with carboxylic acid or alcoholic groups; copolymer waxes synthesised from ethylene and maleic anhydride or from propylene and maleic anhydride; microcrystalline waxes; Fischer-Tropsch waxes; waxes formed from C12-C40 fatty acid esters; natural waxes, such as beeswax, carnauba wax, montan wax and the like; and mixtures thereof.

In the embodiment of the present invention in which the hot melt adhesive formulations disclosed herein comprise at least one wax or a blend of two or more waxes, they comprise from zero to 15% by weight of said wax or of said blend of waxes, preferably from zero to 10% by weight, and even more preferably from zero to 5% by weight, with reference to the total weight of the hot melt adhesive formulation.

### Other Additional Ingredients

The hot melt adhesive formulations according to the present invention can further comprise from 0.01% by weight to 10% by weight of at least one stabiliser, like antioxidants, photo-stabilisers, anti-UV stabilisers and blends thereof. They can also additionally comprise up to a maximum of 15% by weight of optional components, like mineral fillers, pigments, dyes, perfumes, surfactants, antistatic agents.

### EXAMPLES

The present invention is better illustrated by the following examples, which are given merely for the purpose of illustration and are not to be regarded as limiting the scope of the invention or the manner in which it can be practiced. Unless specifically indicated otherwise, parts and percentages are given by weight.

### Examples According to the Invention

### Example 1

The following hot melt adhesive formulation according to the present invention was prepared by blending in the molten state its components at 170° C.

| | % by weight on | |
|---|---|---|
| | the total weight | |
| | of the adhesive | |
| Component | formulation | Nature and supplier |
| Vestoplast | 27.5 | Non-stereospecific |
| 704 | | C2-C3-C4 copolymer |
| | | supplied by Evonik |
| Vestoplast | 27.5 | Non-stereospecific |
| SpotOn30 | | C2-C3-C4 copolymer |
| | | supplied by Evonik |
| Vistamaxx | 1.8 | Thermoplastic |
| 6202 | | elastomer C2-C3 supplied by |
| | | ExxonMobil |
| Regalite | 35.8 | Fully hydrogenated |
| R1100 | | hydrocarbon tackifier |
| | | supplied by Synthomer |
| Primol 352 | 5.0 | Paraffinic mineral |
| | | oil supplied by ExxonMobil |
| Polywax 655 | 0.5 | Polyethylene wax |
| | | supplied by Baker-Hughes |
| | | (M.P. = 99° C.) |
| AC-596 | 0.6 | Polypropylene wax |
| | | modified with maleic |
| | | anhydride supplied by |
| | | Honeywell (M.P. = 141° C.) |
| Irganox 1010 | 1.3 | Antioxidant supplied |
| | | by BASF |

The adhesive formulation of this Example 1 according to the present invention, comprises in particular, as requested by the present invention to enhance and optimise its adhesive properties, both in the dry and wet state, on polar substrates, like cotton, 0.6% by weight of a was modified with polar groups; in this case AC-596 that is a copolymer between propene and maleic anhydride. This hot-melt formulation has a Zero Shear Viscosity at 160° C. equal to 2,390 mPa·s; a Temperature of the Inflection Point of the Tan Delta diagram as a function of temperature, located around the Setting Point, equal to 85.3° C.; a First Crossover Temperature of the rheological moduli Tx, still at Time Zero, equal to 53.5° C.; an Enthalpy of Fusion, after five days of aging, equal to 15.8 J/g; a Yield Stress at 37° C., measured after aging for five days according to the above described method, equal to 0.27 MPa; a Toughness at 37° C., measured after five days according to the same method, equal to 2.55 MJ/m³.

The present adhesive exhibits excellent adhesive properties on fibrous substrates, both under dry and wet conditions. In fact, in the previously described test for the Peel Strength on cotton, the hot melt adhesive of Example 1 exhibits a very high dry Peel Strength, equal to as much as 7.38 N/50 mm; and its Peel Strength remains very good also under wet conditions being at the excellent level of 2.16 N/50 mm. Therefore, comparing its Peel Strengths under dry and wet conditions, it loses, after the absorption of water by the cotton, the 70.7% of its initial dry strength or also retains the 29.3% of it, which is anyhow a surprisingly good result. In fact, this decrease in adhesive strength is still fully acceptable, and the anyhow high absolute value of the Wet Peel Strength assures an optimum resistance even in the presence of large amounts of liquid water and of other aqueous fluids.

The comparisons between the dry Peel Strengths and the corresponding Wet Peel strengths for all the Example, both according to the invention and comparative ones, are also summarised in Table 1 below.

The adhesive formulation of the present Example 1 according to the invention, has also a Brookfield viscosity at 170° C. equal to 2,000 mPa·s and a Ring & Ball softening point equal to 98.9° C.

### Example 2

The following hot melt adhesive formulation according to the present invention was prepared by blending in the molten state its components at 170° C.

| | % by weight on | |
|---|---|---|
| | the total weight | |
| | of the adhesive | |
| Component | formulation | Nature and supplier |
| Eastoflex | 46.3 | Non-stereospecific |
| M1000PL | | C2-C3 copolymer supplied by |
| | | Synthomer |
| Eastotac | 41.5 | Hydrogenated |
| H100L | | hydrocarbon tackifier |
| | | supplied by Synthomer |
| Primol 352 | 7.5 | Paraffinic mineral |
| | | oil supplied by ExxonMobil |
| Polywax 655 | 1.2 | Polyethylene wax |
| | | supplied by Baker-Hughes |
| | | (M.P. = 99° C.) |
| AC-596 | 1.5 | Polypropylene wax |
| | | modified with maleic |
| | | anhydride supplied by |
| | | Honeywell (M.P. = 141° C.) |
| Irganox 1010 | 2.0 | Antioxidant supplied |
| | | by BASF |

The adhesive formulation of the present Example 2 according to the invention, comprises in particular again 1.5% by weight of a wax modified with polar groups; in this case, like in Example 1, AC-596 that is a copolymer between propene and maleic anhydride. This hot-melt formulation of Example 2 exhibits a Zero Shear Viscosity at 160° C. equal to 2,950 mPa·s; a Temperature of the Inflection Point of the Tan Delta diagram as a function of temperature, located around the Setting Point, equal to 71.6° C.; a First Crossover Temperature of the rheological moduli Tx, still at Time Zero, equal to 58.7° C.; an Enthalpy of Fusion, after five days of aging, equal to 16.6 J/g; a Yield Stress at 37° C., measured after aging for five days according to the above described method, equal to 0.35 MPa; a Toughness at 37° C., measured after five days according to the same method, equal to 1.17 MJ/m³.

Also this adhesive has excellent adhesive properties on fibrous substrates, because, in the Peel Strength test as described above, it exhibits a Dry Peel Strength as high as 7.44 N/50 mm. Furthermore, also this adhesive has an excellent retention of said adhesive strength even under wet conditions, because after the absorption of water by the cotton substrate according to the previously described method, it exhibits a Wet Peel Strength which is still at the very good level of 2.95 N/50 mm, i.e. with a decrease of 60.3% versus the corresponding dry value (Table 1).

Moreover, the present adhesive has a Brookfield viscosity at 170° C. equal to 1,660 mPa·s, and a Ring&Ball softening point equal to 107.3° C.

### Example 3

The following hot melt adhesive formulation according to the present invention was prepared by blending in the molten state its components at 170° C.

| | % by weight on | |
|---|---|---|
| | the total weight | |
| | of the adhesive | |
| Component | formulation | Nature and supplier |
| Koattro | 48.8 | Metallocenic |
| PBM1500M | | Polybutene-1 copolymer |
| | | supplied by LyondellBasell |
| Regalite | 42.7 | Fully hydrogenated |
| R1100 | | hydrocarbon tackifier |
| | | supplied by Synthomer |
| Primol 352 | 3.8 | Paraffinic mineral |
| | | oil supplied by ExxonMobil |
| Polywax 655 | 1.2 | Polyethylene wax |
| | | supplied by Baker-Hughes |
| | | (M.P. = 99° C.) |
| AC-596 | 1.5 | Polypropylene wax |
| | | modified with maleic |
| | | anhydride supplied by |
| | | Honeywell (M.P. = 141° C.) |
| Irganox 1010 | 2.0 | Antioxidant supplied |
| | | by BASF |

Also the adhesive formulation of this Example 3 according to the invention, comprises in particular again 1.5% by weight of a wax modified with polar groups; in this case, like in the two previous Examples, AC-596 that is a copolymer between propene and maleic anhydride.

Also the formulation of Example 3 has excellent adhesive properties on fibrous substrates, because, in the Peel Strength test as described above, it exhibits a very high Dry Peel Strength as high as 9.13 N/50 mm. And in addition, also this adhesive has an excellent retention of said adhesive strength even under wet conditions, because after the absorption of water by the cotton substrate according to the previously described method, it exhibits a Wet Peel Strength which is still at the very good level of 3.05 N/50 mm, i.e. with a decrease by 66.7% versus the corresponding dry value (Table 1).

The above formulation of Example 3 also shows a Brookfield viscosity at 170° C. equal to 1,900 mPa·s, and a Ring&Ball softening point equal to 87.4° C.

### Comparative Examples

### Comparative Example 1

The following hot melt adhesive formulation was prepared by blending its components in the molten state at 170° C.

| | % by weight on | |
|---|---|---|
| | the total weight | |
| | of the adhesive | |
| Component | formulation | Nature and supplier |
| Vestoplast | 27.5 | Non-stereospecific |
| 704 | | C2-C3-C4 copolymer |
| | | supplied by Evonik AG |
| Vestoplast | 27.5 | Non-stereospecific |
| SpotOn30 | | C2-C3-C4 copolymer |
| | | supplied by Evonik AG |
| Vistamaxx | 1.8 | Thermoplastic |
| 6202 | | elastomer C2-C3 supplied by |
| | | ExxonMobil |
| Regalite | 35.8 | Fully hydrogenated |
| R1100 | | hydrocarbon tackifier |
| | | supplied by Synthomer |
| Primol 352 | 5.0 | Paraffinic mineral |
| | | oil supplied by ExxonMobil |
| Polywax 2000 | 1.0 | Polyethylene wax |
| | | supplied by Baker-Hughes |
| | | (M.P. = 126° C.) |
| Irganox 1010 | 1.4 | Antioxidant supplied |
| | | by BASF |

The adhesive formulation of Comparative Example 1 is substantially identical to the previous Example 1 according to the invention. However, it does not comprise any wax modified with polar groups, as requested by the present invention to enhance and optimize its adhesive properties, both in the dry and wet state, on polar substrates like cotton. Moreover, it comprises only one wax which has a Drop Melting Point, as measured again according to the method ASTM D 127-87, that is higher than 125° C.

As a consequence, it exhibits poor adhesive properties on a fibrous substrate like cotton, as highlighted also by the fact that it has a Temperature of the Inflection Point of the Tan Delta diagram as a function of temperature, located around the Setting Point, that is excessively high and equal to 100.8° C. Hence, while this hot-melt formulation exhibits a good Dry Peel Strengthequal to 7.37 N/50 mm, after the contact and absorption of water, its Wet Peel Strength drops to the unacceptably low level of 1.1 N/50 mm, that is therefore lower by as much as 85.1% versus the corresponding initial Dry Peel Strength.

This formulation of the Comparative Example 1 shows also the following properties: a Zero Shear Viscosity at 160° C. equal to 3,040 mPa·s; a too high First Crossover Temperature of the rheological moduli Tx, still at Time Zero, equal to 76.7° C.; an Enthalpy of Fusion, after five days of aging, equal to 17.0 J/g; a Yield Stress at 37° C., measured after aging for five days according to the above described method, equal to 0.32 MPa; a Toughness at 37° C., measured after five days according to the same method, equal to 1.45 MJ/m³; a Brookfield viscosity at 170° C. equal to 1,830 mPa·s; and a Ring&Ball softening point of 100.7° C.

### Comparative Example 2

The following hot melt adhesive formulation was prepared by blending in the molten state its components at 170° C.

| | % by weight on | |
|---|---|---|
| | the total weight | |
| | of the adhesive | |
| Component | formulation | Nature and supplier |
| Licocene PP | 71.2 | Metallocenic C2-C3- |
| 2602 | | copolymer supplied by |
| | | Clariant |
| Licocene PP | 19.8 | Metallocenic C2-C3- |
| 1602 | | copolymer supplied by |
| | | Clariant |
| Licocene PP MA | 5.0 | Metallocenic C2-C3- |
| 1332 | | copolymer modified with |
| | | maleic anhydride supplied |
| | | by Clariant |
| Licocene PPA | 3.0 | Amorphous metallocenic |
| 330 | | copolymer C2-C3 |
| | | supplied by Clariant |
| Irganox 1010 | 1.0 | Antioxidant supplied |
| | | by BASF |

Also, this formulation of Comparative Example 2 does not comprise any wax modified with polar groups, as requested by the present invention to enhance and optimize its adhesive properties. Moreover, and differently also from the formulation of the previous Comparative Example 1, the present formulation has also an excessive Enthalpy of Fusion. In fact, after five days of aging, it is as great as 31.2 J/g. It also exhibits a relatively high Zero Shear Viscosity at 160° C., at Time Zero, equal to 6,550 mPa·s while its Brookfield viscosity at 170° C. is equal to 5,300 mPa·s. Moreover, its Temperature of the Inflection Point of the Tan Delta diagram as a function of temperature, located around the Setting Point, and at Time Zero, is equal to 59.5° C.

This comparative formulation 2 shows a Dry Peel Strength equal to 7.1 N/50 mm. However, its Wet Peel Strength drops to unacceptably low values, being as small as just 0.55 N/50 mm, and in this way losing, after contact and absorption of water, as much as 92.3% of its initial Dry Peel Strength.

This formulation further exhibits a First Crossover Temperature of the rheological moduli Tx, at Time Zero, equal to 60.2° C.; a Yield Stress at 37° C., measured after aging for five days according to the above described method, equal to 2.4 MPa; a Toughness at 37° C., measured after five days according to the same method, equal to 1.9 MJ/m³; a Ring&Ball softening point of 94.5° C.

TABLE 1

| | Dry Peel | Wet Peel | |
|---|---|---|---|
| | Strength (N/50 mm) | Strength (N/50 mm) | Delta % |
| EXAMPLE 1 | 7.38 | 2.16 | −70.7% |
| EXAMPLE 2 | 7.44 | 2.95 | −60.3% |
| EXAMPLE 3 | 9.13 | 3.05 | −66.7% |
| COMP. | 7.37 | 1.10 | −85.1% |
| EXAMPLE 1 | | | |
| COMP. | 7.10 | 0.55 | −92.3% |
| EXAMPLE 2 | | | |

1) Use of a hot melt adhesive formulation which exhibits:
a Zero Shear Viscosity not greater than 10,000 mPa·s at 160° C.;
an Enthalpy of Fusion not greater than 30 J/g, after aging for five days;
a Temperature of the Inflection Point of the Tan Delta diagram as a function of temperature, not higher than 95° C., said Inflection Point being located around the Setting Point, as measured at Time Zero and in decreasing temperature, according to the method described herein;
said hot melt adhesive formulation further exhibiting:
Dry Peel Strength, after aging for five days, not lower than 2.0 N per 50 mm width;
Wet Peel Strength after water absorption and after aging for five days, which differs from the corresponding Dry Peel Strength by no more than 80%;
wherein such Peel Strengths are measured according to the methods described herein,
in manufacturing of absorbent hygienic articles, which contain at least one fibrous substrate, said fibrous substrate being either a woven or a non-woven one, and its fibres being either natural or synthetic ones, either hydrophobic or hydrophilic, wherein the hot melt adhesive formulation includes at least one wax or a mixture of waxes synthesised from ethylene and maleic anhydride or from propylene and maleic anhydride or C12-C40 hydrocarbon waxes, in their versions modified with carboxylic acid or alcoholic groups, said wax or waxes having a “Drop Melting Point” in the range from 40° C. to 170° C., as measured according to the ASTM D 127-87 method.

2) Use of the hot-melt adhesive formulation according to claim 1, wherein the polymer or mixture of mutually constitute from 5% to 99.5% by weight of the total weight of the adhesive formulation itself.

3) Use of the hot melt adhesive formulation according to claim 1, wherein the hot melt adhesive formulation has a First Crossover Temperature of the rheological modules not higher than 75° C., as measured at Time Zero and in decreasing temperature, according to the method described herein.

4) Use of the hot melt adhesive formulation according to claim 1, wherein the hot melt adhesive formulation comprises at least one tackifier or a mixture of tackifiers having a Ring & Ball Softening Temperature ranging from 5° C. to 160° C. as measured according to the method described herein.

5) Use of the hot melt adhesive formulation according to claim 4, wherein the tackifier or the mixture of tackifiers are selected from aliphatic hydrocarbon tackifiers, and their partially or totally hydrogenated derivatives; aromatic hydrocarbon tackifiers and their partially or totally hydrogenated derivatives; aliphatic/aromatic hydrocarbon tackifiers, and their partially or totally hydrogenated derivatives; modified polyterpene and terpene tackifiers, and their partially or totally hydrogenated derivatives; Rosins, their esters, and their partially or totally hydrogenated derivatives; and mixtures thereof.

6) Use of the hot melt adhesive formulation according to claim 4, wherein the tackifier or mixture of tackifiers constitute from zero to 80% by weight of the adhesive formulation.

7) Use of the hot melt adhesive formulation according to claim 1, wherein the hot melt adhesive formulation comprises at least one plasticiser or a mixture of plasticisers which are liquid at room temperature.

8) Use of the hot melt adhesive formulation according to claim 7, wherein the plasticiser or mixture of plasticisers which are liquid at room temperature are selected from paraffinic mineral oils and naphtenic mineral oils and mixtures thereof; paraffinic and naphtenic hydrocarbons which are liquid at room temperature, and mixtures thereof; polyolefin oligomers which are liquid at room temperature, and copolymers thereof, and mixtures thereof; plasticisers which are liquid at room temperature formed by esters; natural and synthetic fats; vegetable oils; and mixtures thereof.

9) Use of the hot melt adhesive formulation according to claim 7, wherein the plasticizer or mixture of plasticisers which are liquid at room temperature consist of an oligomer or a mixture of synthetic poly-alpha-olefin oligomers, synthesized from C2-C20 olefins and having a Number Average Molecular Weight Mn in the range from 150 to 15,000 g/mole.

10) Use of the hot melt adhesive formulation according to claim 7, wherein the plasticiser, liquid at room temperature, or the mixture of plasticisers which is liquid at room temperature, constitutes from zero to 40% by weight of the total weight of the adhesive formulation.

11) Use of the hot melt adhesive formulation according to claim 1, wherein the wax or mixture of waxes constitutes from zero to 15% by weight of the total weight of the adhesive formulation.

12) Use of the hot melt adhesive formulation according to claim 1, wherein the hot melt adhesive formulation has a Brookfield viscosity at 170° C. not greater than 15,000 mPa·s as measured according to the ASTM D3236-88 method.

13) Use of the hot melt adhesive formulation according to claim 1, wherein the hot melt adhesive formulation has a Ring & Ball Softening Temperature not higher than 135° C. as measured according to the method described herein.

14) Hot melt adhesive formulation that comprises at least one polymer, that can be either a homopolymer or a copolymer, or a mixture of two or more polymers, that are mutually compatible and that can be either homopolymers or copolymers, where said polymer or mixture of polymers is selected from homopolymers of a C2-C12 olefin or of a C4-C12 diolefin; copolymers between the same olefins and diolefins; copolymers between a C2-C12 olefin and vinyl or acrylic monomers; styrenic block-copolymers both in the non-hydrogenated and fully hydrogenated state; and mixtures thereof, said formulation comprising at least one wax or a mixture of waxes synthesized from ethylene and maleic anhydride or from propylene and maleic anhydride or C12-C40 hydrocarbon waxes, in their versions modified with carboxylic acid or alcoholic groups, said wax or waxes having a “Drop Melting Point” in the range from 40° C. to 170° C., as measured according to the ASTM D 127-87 method,
said hot melt adhesive formulation being characterized in that it shows:
a Zero Shear Viscosity at 160° C. not greater than 10,000 mPa·s, as measured according to the herein described method;
an Enthalpy of Fusion, after aging for five days, not greater than 30 J/g, as measured according to the herein described method;
a Temperature of the Inflection Point of the Tan Delta diagram as a function of temperature, not higher than 95° C., said Inflection Point being located around the Setting Point, as measured at Time Zero and in decreasing temperature, according to the method described herein;
said hot-melt adhesive formulation being further characterised in that it has:
a Dry Peel Strength, after aging for five days, not lower than 2.0 N per 50 mm width;
a Wet Peel Strength after water absorption and after aging for five days, which differs from the corresponding Dry Peel Strength by no more than 80%;
wherein such Peel Strengths are measured according to the methods described herein,
said hot melt formulation for the adhesion to fibrous substrates having also:
a Yield Stress not lower than 0.1 MPa after aging for five days, as measured at a temperature of 37° C., according to the method described herein; and
having a Toughness not lower than 0.5 MJ/m³ after aging for five days, as measured at a temperature of 37° C., according to the method described herein.

15) Bonded structure including:
a first substrate;
a second substrate; and
a hot melt adhesive formulation as in claim 14, bonding the first and second substrate together, which, when applied at a basis weight in the range from 0.5 to 50 g/m², provides the bonded structure with a Dry Peel Strength greater than 0.25 N per 50 mm of width, as measured according to the method described herein.

16) Bonded structure of claim 15, wherein at least one of the bonded substrates is a fibrous substrate, both a woven or non-woven one.

17) Bonded structure of claim 16, wherein at least one fibrous, woven or non-woven substrate includes at least 50% by weight of natural or artificial cellulosic fibers.

18) Hygienic absorbent article which includes the hot melt adhesive formulation of claim 14.

19) Hygienic absorbent article which includes a bonded structure according to claim 15.

20) Hygienic absorbent article of claim 18, wherein said article is an absorbent diaper for babies or children, training pants for toddlers, an absorbent diaper for incontinent adults, or an absorbent article for feminine hygiene.

21) Hygienic absorbent article according to claim 18, wherein the hot melt adhesive formulation is used for at least one of the following uses: i) as a general adhesive for the construction of the whole article; ii) for bonding elastic components; iii) for consolidating and ensuring, even in use, the integrity of the absorbent layers of the absorbent hygienic article; or iv) for bonding perforated films having either a two-dimensional or a three-dimensional structure.

22) An article including the hot-melt adhesive formulation of claim 14, wherein said article is an absorbent mattress, an absorbent sheet, an absorbent surgical laminate for medical use, or a product for covering and protecting wounds.

23) An article including the hot-melt adhesive formulation of claim 14, wherein said article is a mattress or a component thereof.

24) An article including the hot-melt adhesive formulation of claim 14, wherein said article is a packaging article.
